(19) United States
(12) Patent Application Publication
(10) Pub. No.: US 2006/0263908 A1
(43) Pub. Date: Nov. 23, 2006

(54) Fluorescent complex, a fluorescent particle and a fluorescence detection method

(75) Inventor: Hiroyuki Hirai, Kanagawa (JP)

Correspondence Address: TAIYO CORPORATION, 401 HOLLAND LANE, #407, ALEXANDRIA, VA, 22314, US

(73) Assignee: FUJI PHOTO FILM CO., LTD.

(21) Appl. No.: 11/192,361

(22) Filed: Jul. 29, 2005

(30) Foreign Application Priority Data
Mar. 8, 2004 (JP) 2004-227208
Mar. 8, 2004 (JP) 2004-227107
Jul. 25, 2005 (JP) 2005-214739
Jul. 25, 2005 (JP) 2005-214740

(51) Int. Cl. G01N 33/553; G01N 33/543

(52) U.S. Cl. 436/526; 436/518

(57) ABSTRACT

The fluorescent complexes of the invention each comprise a magnetic nano-particle; an inorganic phosphor nano-particle; and a linker, wherein the magnetic nano-particles have an average particle diameter of 2 to 100 nm, the inorganic phosphor nano-particles have an average particle diameter of 1 to 50 nm, and the linker links the magnetic nano-particles with the inorganic phosphor nano-particles. The fluorescence detection method of the present invention comprises detecting a target substance in a sample by using the fluorescent complex of the present invention.

### CROSS-REFERENCE TO RELATED APPLICATION

This application claims priority under 35 USC 119 from Japanese Patent Applications Nos. 2004-227208, 2004-227107, 2005-214740 and 2005-214739 the disclosures of which are incorporated by reference herein.

### BACKGROUND OF THE INVENTION

1. Field of the Invention

The present invention relates to a fluorescent complex and a fluorescence detection method, and particularly relates to a fluorescent complex and a fluorescence detection method which use a nano-particle.

2. Description of the Related Art

It is known that particle materials of nano size exhibit properties different from that of the bulk materials. For example, with a nano-scale semiconductor, the band gap, which has conventionally been considered to be material-specific, varies depending upon the size of the particle, which is well known as the so-called quantum size effect. The size at which this effect becomes significant varies depending upon the type of semiconductor material, being typically a few dozen nm or less. Therefore, single order nano-particles are particularly important. In addition, it is known that some materials exhibit such effects as, simultaneously with this quantum size effect becoming significant, the life of the fluorescence becomes shorter, or light emission which has not been observed until that time becomes possible to be observed, and the like. Thus, because nano-size, and particularly single order nano-size materials have properties which are different from those of conventionally known bulk materials, they are attracting great attention of the scientists and engineers.

For example, semiconductor nano-particle fluorescent materials with which semiconductor nano-particles, such as CdSe/CdS (core/shell), CdSe/ZnS (core/shell), and the like, are used, and where to the surface of these semiconductor nano-particles or the surface of the beads containing these semiconductor nano-particles a molecular probe is bound for detection of the target molecule have been proposed (as given in Science, 1998, vol. 281, No. 25, pp. 2013 to 2016; and Nature Biotechnology, 2001, vol. 19, pp. 631 to 635). By providing these semiconductor nano-particles with different crystallite sizes, they can emit light at different wavelengths. In addition, by using plural labeling beads which combine specific light emission wavelengths with particular light emission strengths, simultaneous multiple tests can be carried out. Semiconductor nano-particle fluorescent materials provide excellent features as labeling materials, such as that they are highly sensitive, low-cost, and easy to be automated, and the like. Therefore, by using a semiconductor nano-particle fluorescent material as a labeling material, it has been made possible to detect a specific site in the living body, a certain substance in the blood plasma, and the like with high sensitivity and high speed.

In recent years, as means for efficiently collecting target substances, the use of magnetic fine particles has been proposed. Because magnetic fine particles can be conveniently and efficiently collected by using an external magnetic field, they have been used as accurate detection means for detecting substances from living organisms, and the like (for example, WO00/05357, and Japanese Patent Application Laid-Open (JP-A) No. 5-292971). Likewise, as means for efficiently separating small molecules, methods which utilize macromolecules having a lower critical solution temperature (LCST) and an upper critical solution temperature (UCST) have been proposed (for example, WO02/16571 and WO02/16528 pamphlets, and JP-A No. 2002-60436).

In the research fields of biochemistry and medical science, exact detection of complicated vital phenomena has been demanded. Especially in vital phenomena, even a trace amount of substance in the cell often plays an important role, and it is necessary to accurately capture such a trace amount of a substance by entering into the cell.

However, in order to detect with certainty a trace amount of substance (the target substance) existing in the sample, repetition of accurate detection procedures may be conceived, but when only a small amount of sample is used, it is required to minimize the losses due to repeating the detection procedure. In addition, repeating the same operation takes much labor and long time.

Thus, the purpose of the present invention is to provide a fluorescent complex and a fluorescence detection method for efficiently and certainly detecting the target substance, even if it exists in a trace amount in a minute region, as well as a fluorescent particle which can be used for these.

### SUMMARY OF THE INVENTION

A first aspect of the present invention provides fluorescent complexes, each comprising a magnetic nano-particle; an inorganic phosphor nano-particle; and a linker; wherein the magnetic nano-particles have an average particle diameter of 2 to 100 nm, the inorganic phosphor nano-particles have an average particle diameter of 1 to 50 nm, and the linker links the magnetic nano-particles with the inorganic phosphor nano-particles.

A second aspect of the present invention provides fluorescent particles each comprising an inorganic phosphor nano-particle, and an external stimuli responsive compound, wherein the inorganic phosphor nano-particles have an average particle diameter of 1 to 50 nm and the external stimuli responsive compound is disposed on the surface of the inorganic phosphor nano-particles. Herein, the composition of said inorganic phosphor nano-particles, and the light emission characteristics thereof, such as, the light emission wavelength, the excitation wavelength, the half width for light emission, and the like, are not restricted, and any inorganic phosphor nano-particles can be used. Particularly, inorganic phosphor nano-particles which consist of elements friendly to the human body and the environment, which have excitation wavelengths in the near-ultraviolet light region at 300 to 410 nm or so, and which have light emission wavelengths in the visible region are preferable. As such inorganic phosphor nano-particles, which are constituted by the group 12 with group 16 elements, particularly those composed from ZnS or ZnO; those which are constituted by the group 13 with group 15 elements, particularly those composed from GaN, InN, GaInN, AlInN or the like; and those which are constituted by the group 2 elements with group 14 and group 16 elements, particularly those from multiple oxides, such as CaSiO₃, SrSiO₃, Ca₂SiO₄, Sr₂SiO₄, CaMgSiO₄ or the like, are preferable. These compounds may be doped with rare earth ions, such as Eu, Ce, Th, or the like, or metallic ion, such as Mn, Cu, or the like, as required.

In addition, it is preferable that said magnetic nano-particles be an iron oxide or a ferrite.

Further, said linker preferably comprises an external stimuli responsive compound.

In addition, said fluorescent complex or said fluorescent particle preferably has a ligand for binding to the target substance.

A third aspect of the present invention provides a fluorescence detecting method for detecting a target substance in a sample using a phosphor, comprising: combining the fluorescent complexes of the invention, that further comprise a ligand for causing the target substance to bind thereto, with the sample for formation in the sample of fluorescent complexes to which the target substance has been bound; applying an external magnetic field to the fluorescent complexes in the sample for collecting the fluorescent complexes; irradiating the collected fluorescent complexes with excitation light for exciting the phosphor nano-particles to cause the phosphor nano-particles to emit fluorescent light; detecting the fluorescence emitted from the fluorescent complex; and detecting the target substance in the sample on the basis of the fluorescence emission.

Herein, an external stimuli responsive compound may be disposed on the surface of either the magnetic nano-particle or the inorganic phosphor nano-particle or both.

A fourth aspect of the present invention provides a fluorescence detecting method for detecting a target substance in a sample using a phosphor, comprising: combining magnetic nano-particles, comprising a ligand for causing the target substance to bind thereto and having an average particle diameter of 2 to 100 nm, with the sample for formation in the sample of magnetic nano-particles to which the target substance has been bound; applying an external magnetic field to the magnetic nano-particles in the sample for collecting the magnetic nano-particles to which the target substance has been bound; combining inorganic phosphor nano-particles, which can be mutually bound to the magnetic nano-particles through a linker and which have an average particle diameter of 1 to 50 nm, with the collected magnetic nano-particles for formation of fluorescent complexes which are the fluorescent complexes of claim 1, and to which the target substance has been bound; irradiating the fluorescent complexes with excitation light for exciting the phosphor nano-particles to cause the phosphor nano-particles to emit fluorescent light; detecting the fluorescence emitted from the fluorescent complexes; and detecting the target substance in the sample on the basis of the fluorescence emission. Herein, an external stimuli responsive compound may be disposed on the surface of either the magnetic nano-particle or the inorganic phosphor nano-particle.

A fifth aspect of the present invention provides a fluorescence detection method for detecting a target substance in a sample using a phosphor, comprising: combining the fluorescent particles of the invention, that further comprise a ligand for causing the target substance to bind thereto with the sample for formation of fluorescent particles to which the target substance has been bound; applying an external stimulus for aggregating the fluorescent particles; irradiating the phosphor nano-particles with excitation light for exciting it to cause the phosphor nano-particles to emit fluorescent light; detecting the fluorescence emission from the fluorescent particles; and detecting the target substance in the sample on the basis of the fluorescence emission.

In the present invention, the term “fluorescent complex” means a structure which is configured by an inorganic phosphor nano-particle and a magnetic nano-particle, which are each defined in the present specification, through a linker.

Further, in the present invention, the term “fluorescent particle” means a structure which is configured by an inorganic phosphor nano-particle and an external stimuli responsive compound disposed on the surface thereof, which are each defined in the present specification.

### DETAILED DESCRIPTION OF THE INVENTION

The present invention will be hereinafter described in detail.

(A) Fluorescent Complex

The fluorescent complex of the present invention comprises a magnetic nano-particle having an average particle diameter of 2 to 100 nm, an inorganic phosphor nano-particle having an average particle diameter of 1 to 50 nm, and a linker which conjugates said magnetic nano-particle with said inorganic phosphor nano-particle.

According to the present invention, the fluorescent complex which is used for detecting a target substance comprises a magnetic nano-particle, and thus by using an external magnetic force, the fluorescent complex to which the target substance has been bound can be easily collected. In addition, the fluorescent complex comprises an inorganic phosphor nano-particle, and thus the target substance can be detected with high sensitivity. Thereby, even if the target substance in the sample exists in a minute region such as in a cell, or the like, the target substances can be efficiently collected with a magnetic force to be certainly and efficiently detected.

Inorganic Phosphor Nano-Particle

The phosphor nano-particle in the present invention is an inorganic particle which emits fluorescence with prescribed excitation light, and the average particle diameter is 1 to 50 nm; is preferably 1 to 20 nm; and is more preferably 1 to 10 nm. If the number-average particle diameter is 1 nm or more, the stability of the phosphor nano-particle is good, and if the number-average particle diameter is 50 nm or less, the light scatter is low in detection of the target substance, and the dispersibility of the particles is good, which allows the target substance to be detected with a high sensitivity, and makes it easy for the particle to enter into a cell.

The coefficient of variation of particle diameter distribution for the phosphor nano-particle is preferably 0 to 50%; is more preferably 0 to 20%; and is still more preferably 0 to 10%. The coefficient of variation means the arithmetic standard deviation divided by the number-average particle diameter, and expressed as a percentage (i.e., arithmetic standard deviation x 100/number-average particle diameter).

In the present invention, the composition of said inorganic phosphor nano-particle, and the light emission characteristics thereof, such as, the light emission wavelength, the excitation wavelength, the half width for light emission, and the like, are not restricted, and any inorganic phosphor nano-particle can be used.

Preferable phosphor nano-particles are a phosphor nano-particle of metal oxide or metal sulfide. Examples of the metal constituting a metal oxide or a metal sulfide include group 12, such as Zn, and the like; group 3, such as Y, Eu, Th, and the like; group 13, such as Ga, In, and the like; group 4, such as Zr, Hf, and the like; group 14, such as Si, Ge, and the like; group 5, such as V, Nb, and the like; group 6, such as Mo, W, and the like; and the like. Among these, Zn, which exhibits no high reactivity to a living body is particularly preferable. In addition, as such an inorganic phosphor nano-particle, that which is constituted by the group 12 with group 16 elements, particularly those composed of ZnS or ZnO; and that which is constituted by the group 13 with group 15 elements, particularly those composed of GaN, InN, GaInN, AlInN, or the like, can be mentioned as preferable. In addition, such inorganic phosphor nano-particles may be multiple metal oxides which are constituted by elements belonging to three or more groups, and examples thereof include Zn₂SiO₄, CaSiO₃, Ca₂SiO₄, SrSiO₃, Sr₂SiO₄, MgWO₄, YVO₄, Y₂SiO₅, SrAl₂O₄, Y₃Al₅O₁₂, and the like. Among these, those which are constituted by group 2 elements with group 14 and group 16 elements, particularly the multiple oxides, such as CaSiO₃, SrSiO₃, Ca₂SiO₄, Sr₂SiO₄, CaMgSiO₄, and the like, are preferable.

As the phosphor nano-particle in the present invention, it is particularly preferable that the phosphor nano-particle be made of zinc oxide (ZnO) or zinc sulfide (ZnS), and it is most preferable that the phosphor nano-particle be made of zinc oxide (ZnO), from the viewpoints of that it can be stably manufactured; the possibility of toxicity is less; it can be manufactured at low cost; the monodispersibility of the particles is high; intense light emission can be obtained; the wavelength region of the emission spectrum tends to match the present purpose; the excitation light has a wavelength in the visible to near-ultraviolet region.

Further, it is also preferable that the phosphor nano-particle of these metal oxides (multiple metal oxides) or metal sulfides contain a small amount of a metallic ion which is different from the constituent metal in the metal oxide or metal sulfide. Examples of metallic ions include rare earth ions and metallic ions, such as Mn, Cu, Eu, Th, Tm, Ce, Al, Ag, and the like, and Mn and Eu are preferable from the viewpoints of that the visibility is high of their light generation and they can be stably manufactured. It is also preferable that these metallic ions be doped as compounds resulting from combination with a chloride ion or a fluoride ion. The doping metallic ion may be of one type of atom or of a plurality of types of atom. Therefore, examples of the phosphor nano-particle containing such a metallic ion(s) include ZnS:Mn, ZnO:Eu, and the like. The concentration of the metallic ions varies in optimum amount, depending upon the metal constituting the phosphor nano-particle and the type thereof, however, a concentration in the range of 0.001 to 10 atomic percent is preferable; and the range of 0.01 to 10 atomic percent is more preferable.

From the viewpoints of separation between the excitation light and the signal fluorescence, the utilization of a low-cost light source, and the construction of a convenient detection system, the phosphor nano-particle pertaining to the present invention is preferably excited with light in the near-ultraviolet region; more preferably uses 300 nm to 410 nm near-ultraviolet light as the excitation light; and with this excitation light, emits light preferably in the visible region, and more preferably visible light in the region of 400 nm to 700 nm. When a fluorescent coloring matter is further bound to the surface of the phosphor nano-particle, as described later, emission of the visible light allows the fluorescent coloring matter in a different visible region to be excited by energy transfer, and thus the fluorescent coloring matter can be caused to develop the color with less energy, and particularly with no high reactivity thereto by a living body.

The half width for light emission of the phosphor nano-particle is preferably 50 to 200 nm, and in order to detect the light emission highly sensitively with a simple apparatus, it is preferably 60 to 200 nm. Further, as a fluorescent labeling material, it is preferable that the light emission peak wavelength and the absorption peak wavelength be different from each other, and in order to detect the light emission with a high sensitivity, the light emission peak wavelength is more preferably separated from the edge of the absorption wavelength by 20 nm or more; and particularly preferably by 50 nm or more. Phosphor nano-particles having such a peak wavelength and half width for light emission are the phosphor nano-particle of metal oxides or metal sulfides, and persons skilled in the art can easily obtain them by appropriately selecting the constituent metal and the like, as described above.

In addition, when fluorescent coloring matter is bound to the surface of the phosphor nano-particle in the present invention, the phosphor nano-particle is preferably a particle which can provide energy for the fluorescent coloring matter by light emission. Thereby, a plurality of fluorescent coloring matters which are excited at different energy levels can be caused to emit light at the same time.

Further, the phosphor nano-particle of metal oxide or metal sulfide of the present invention is preferably a phosphor nano-particle of a metal oxide or metal sulfide which is excellent in coatability with the surface modifying agent later described.

Surface Modifying Agent

The phosphor nano-particle in the present invention is preferably that which is surface-modified by using a compound represented by the following formula [I] or a decomposition product thereof as a surface modifying agent. This is particularly preferable when the phosphor nano-particle is a phosphor nano-particle of metal oxide or metal sulfide. Thereby, a high sensitivity can be provided with the dispersibility of the phosphor nano-particles in water or a hydrophilic solvent can be improved, and the elution of the phosphor nano-particle or the extinction of the fluorescence by body fluids can be prevented. In addition, there arise advantages that the light emission characteristics are uniform with a broad half width, and that functionalization, such as by binding a molecular probe (a ligand) for detecting the target molecule, or the like, can be easily performed.
M-(R)₄ formula [I]

(In the formula, M denotes a Si or Ti atom, and R an organic group. Rs may be the same or different, respectively, but at least one of the Rs denotes a group having a reactivity to the linker or the ligand later described.)

Among the organic groups denoted by R, examples of groups having a reactivity to linkers or ligands include those to the end of which a vinyl group, an allyloxy group, an acryloyl group, a methacryloyl group, an isocyanate group, a formyl group, an epoxy group, a maleimide group, a mercapto group, an amino group, a carboxyl group, halogen, or the like is connected through a conjugating group. Among these groups having a reactivity, groups having an amino group at the end are particularly preferable. The linker in the present invention will be later described.

Examples of the conjugating group include alkylene groups (for example, the methylene group, the ethylene group, the trimethylene group, the tetramethylene group, the propylene group, the ethylethylene group, the cyclohexylene group, and the like each having 1 to 10 carbon atoms, preferably I to 8 carbon atoms in a chainlike or cyclic form).

The conjugating group may have an unsaturated bond. Examples of the unsaturated group include alkenylene groups (for example, the vinylene group, the propenylene group, the 1-butenylene group, the 2-butenylene group, the 2-pentenylene group, the 8-hexadecenylene group, the 1,3-butanedienylene group, the cyclohexenylene group, and the like each having 1 to 10 carbon atoms, preferably 1 to 8 carbon atoms in a chainlike or cyclic form), arylene groups (for example, the phenylene group, the naphthylene group and the like each having 6 to 10 carbon atoms, preferably the phenylene group having 6 carbon atoms).

The conjugating group may have one or two or more hetero atoms (hetero atom means any atom other than a carbon atom, such as a nitrogen atom, oxygen atom, sulfur atom, or the like). A hetero atom is preferably an oxygen atom or sulfur atom, and is more preferably an oxygen atom. The number of hetero atoms is not particularly specified, but 5 or less is preferable, and 3 or less is the most preferable.

The conjugating group may contain, as a partial structure, a functional group containing a carbon atom adjacent to the above-mentioned hetero atom. Examples of functional groups include ester groups (including carboxylate esters, carbonate esters, sulfonate esters, and sulfinate esters), amide groups (including carboxylamide, urethane, sulfonamide, and sulfinamide), ether groups, thioether groups, disulfide groups, amino groups, imide groups, and the like. The above-mentioned functional groups may have a further substituent, and the conjugating group may have a plurality of these respective functional groups. When the conjugating group has a plurality of these functional groups, they can be either the same or different from each other.

Preferable functional groups are an alkenyl group, an ester group, an amide group, an ether group, a thioether group, a disulfide group, or an amino group, and more preferably an alkenyl group, an ester group, or an ether group.

As the other organic groups denoted by R, arbitrary groups may be mentioned, however, alkoxy groups, such as methoxy group, ethoxy group, isopropoxy group, n-propoxy group, t-butoxy group, n-butoxy group, and the like, and the phenoxy groups are preferable. These alkoxy groups and phenoxy groups may have a further substituent, however, it is preferable that the total number of carbon atoms be 0.8 or less. The surface modifying agent for use with the present invention may be one in which an amino group, carboxylic group, or the like forms a salt with an acid or base.

The decomposition products of surface modifying agents represented by the formula [I] for use with the present invention refer to hydroxides obtained by the hydrolysis of an alkoxy group, a low-molecular weight oligomer generated by a dehydration condensation reaction between hydroxyl groups (which may be any of linear, cyclic, or cross-linking structures, and the like), dealcohol condensation reaction products by reaction between hydroxyl groups and unhydrolyzed alkoxy groups, and sols and gels formed by further dehydration condensation reaction of these.

Specific examples of the surface modifying agent represented by the formula [I] for use with the present invention are as follows, but not limited to these compounds in the present invention.

N-(2-aminoethyl)-3-aminopropylmethyldimethoxysilane, N-(2-aminoethyl)-3-aminopropyltrimethoxysilane, N-(2-aminoethyl)-3-aminopropyltriethoxysilane, 3-aminopropyltrimethoxysilane, aminophenyltrimethoxysilane, 3-aminopropyltriethoxysilane, bis(trimethoxysilylpropyl)amine, N-(3-aminopropyl)-benzamidetrimethoxysilane, 3-hydrazidepropyltrimethoxysilane, 3-maleimidepropyltrimethoxysilane, (p-carboxy)phenyltrimethoxysilane, 3-carboxypropyltrimethoxysilane, 3-aminopropyltitaniumtripropoxide, 3-aminopropylmethoxyethyltitaniumdiethoxide, 3-carboxypropyltitaniumtrimethoxide, and the like.

The surface modifying agent for use with the present invention may be one in which a terminal NH₂ group or COOH group forms a salt with an acid or base.

When the phosphor nano-particle in the present invention is a phosphor nano-particle of a metal sulfide, it is also preferable to use a compound represented by the following formula [II] as the surface modifying agent.
HS-L-W formula [II]

In the formula, L denotes the above-mentioned bivalent conjugating group, and W denotes COOZ or NH₂. Herein, Z denotes a hydrogen atom, an alkaline metal atom, or NX₄, where X denotes a hydrogen atom or an alkyl group.

When W is NH₂, the compound may have formed a salt with hydrochloric acid, sulfuric acid, nitric acid, phosphoric acid, sulfonic acid, or the like. As the alkaline metal atom denoted by Z, lithium (Li), sodium (Na), potassium (K), and the like can be mentioned, and as the alkyl group denoted by X, the methyl group, the ethyl group, the n-propyl group, the isopropyl group, the t-butyl group, the octyl group, the cetyl group, and the like each having 1 to 20 carbon atoms, preferably 1 to 18 carbon atoms in the chainlike form can be mentioned. The four Xs may be either the same or different.

Specific examples of the surface modifying agent represented by the formula [II] include mercaptoacetic acid, 2-mercaptopropionic acid, 3-mercaptopropionic acid, 2-mercaptobutyric acid, 4-mercaptobutyric acid, 8-mercaptooctanoic acid, 11-mercaptoundecanoic acid, 18-mercaptostearic acid, 3-mercaptoacrylic acid, mercaptomethacrylic acid, 4-mercaptocrotonic acid, 18-mercaptooleic acid, thiomalic acid, mercaptopropiol acid, 4-mercaptophenylhydrocinnamic acid, 2-mercaptoethylamine, 2-mercaptopropylamine, 3-mercaptopropylamine, 3-mercapto-n-butylamine, 4-mercapto-n-butylamine, 2-mercapto-t-butylamine, 8-mercaptooctylamine, 11-mercaptoundecylamine, 18-mercaptostearylamine, 18-mercaptooleylamine, 5-aminopentanoicacid(2-mercapto-ethyl)-amide, 6-aminohexanoicacid(2-mercapto-ethyl)-amide, 11-aminoundecanoicacid(2-mercapto-ethyl)-amide, 5-aminopentanoicacid-3-mercapto-propylester, 11-aminoundecanoicacid-3-mercaptopropylester, 3-(1-amino-undecyloxy)-propane-1-thiol, (2-mercapto-ethylamino)-aceticacid-2-[2-(2-aminoacetoxy)-ethoxy]-ethylester, and the like. The compound having an amino group may be one which has formed a salt with an acid, as described above. With respect to the present invention, the surface modifying agent is not limited to these.

The surface modifying agents which can be used with the present invention may coat the entire surface of the phosphor nano-particle or may be bound to a part thereof. In addition, in the present invention, the surface modifying agent may be used alone or in combination with another.

In the present invention, the surface modifying agents may be part of the linker.

In the present invention, in addition to the above-mentioned surface modifying agent, well-known surface modifying agents (for example, polyethylene glycol, polyoxyethylene(l)laurylether phosphoric acid, laurylether phosphoric acid, trioctyl phosphine, trioctyl phosphine oxide, sodium polyphosphate, bis(2-ethylhexyl)sodium sulfosuccinate, or the like) may coexist during or after the nano-particle synthesis.

Manufacturing Method For Phosphor Nano-Particle

The inorganic phosphor nano-particle can be manufactured by using a well-known synthesizing method. For example, a liquid phase synthesizing methods, such as homogeneous solution precipitation (co-precipitation) method, the reverse micelle method (microemulsion method), the hot-soap method, the sol-gel method, the Solvothermal method, the molten urea method, the metal complex method, or the like; gas phase synthesizing methods, such as the CVD method, the sputtering method, the laser ablation method, the Joule-Quench™ method, the gas vapor deposition method, or the like; or special synthesizing methods, such as the spray pyrolysis method, the supercritical method, or the like can be applied. In addition, these synthesizing methods can be combined with one another for use. For liquid phase synthesizing methods, microwave irradiation, ultrasonic wave irradiation, or the like may be used in conjunction therewith, and a minute reaction space, such as that of a microreactor, or the like, may be utilized.

In the present invention, the inorganic phosphor nano-particles are required to be colloidally dispersed, and it is preferable that calcination, which is generally used for manufacturing fluorescent materials, is not carried out. In addition, in order to control crystal growth and aggregation of particles, it is preferable that reaction be undertaken in the presence of an appropriate surface modifying agent or by using a micro or nano space.

The metal oxide phosphor nano-particle pertaining to the present invention can be obtained by: a liquid synthesizing method, such as the sol-gel method, which hydrolyzes an organic metal compound, such as an alkoxide, an acetylacetonate, or the like, of the metal; the hydroxide precipitation method, which adds an alkali to the aqueous solution of a salt of the metal to precipitate it as a hydroxide before dehydration and annealing; the ultrasonic wave decomposition method, which subjects a solution of an above-mentioned precursor of the metal to irradiation by ultrasound; the solvothermal method, which carries out decomposition reaction at high temperature and high voltage; and the spray pyrolysis, which carries out spraying at high temperature, or the like. Further, it can also be obtained by a gas synthesizing method, such as the thermal CVD method or the plasma CVD method, which uses an organic metal compound; the sputtering method, which uses a target of the metal or an oxide of the metal; or the laser ablation method, or the like.

Metal sulfide phosphor nano-particles pertaining to the present invention can be obtained by a liquid synthesizing method, such as the hot-soap method, which causes crystal growth of a pyrolyzable metal compound, such as a diethyldithiocarbamate compound, or the like, of the metal in the high-boiling point organic solvent, such as a trialkylphosphineoxide, a trialkylphosphine, an ω-aminoalkane, or the like; the co-precipitation method, which adds the solution of a sulfide, such as sodium sulfide, ammonium sulfide or the like, to a solution of a salt of the metal to cause crystal growth; or the reverse micelle method, which causes an aqueous solution of the above-mentioned raw material containing a surface-active agent to exist in a non-polar organic solvent, such as an alkane, an ether, an aromatic hydrocarbon, or the like, as a reverse micelle, and causes crystal growth in the reverse micelle. In addition, they can also be obtained by the same gas phase synthesizing method as for the metal oxide phosphor nano-particles.

The surface modifying agent represented by the formula [I] that is used with the present invention can be added during synthesis of the phosphor nano-particle, however, preferably, it is added after synthesis, and by hydrolyzing at least a part thereof, it is caused to bind to the phosphor nano-particle, coating (surface modifying) at least a part of the surface of the nano-particle. The phosphor nano-particles may be cleaned and refined by a conventional method, such as centrifugal separation, filtering, or the like, before being dispersed in a solvent (preferably, a hydrophilic organic solvent, such as methanol, ethanol, isopropylalcohol, 2-ethoxyethanol, or the like) containing the surface modifying agent used with the present invention for coating.

The amount of addition of the surface modifying agent used with the present invention varies depending upon the particle size, the concentration of the particles, the type (size and structure) of the surface modifying agent, and the like, however, relative to a metal oxide or a metal sulfide, the amount of addition is preferably 0.001 to 10 times the molar quantity, and is more preferably 0.01 to 2 times the molar quantity.

In the present invention, a known surface modifying agent may be used in conjunction. The amount of addition of a known surface modifying agent is not particularly limited, but relative to the surface modifying agent represented by the formula [I] or [II], the amount of addition is preferably 0.01 to 100 times the molar quantity, and is more preferably 0.05 to 10 times the molar quantity.

The phosphor nano-particles to which a surface modifying agent is bound may provide a dispersion solution in which they are dispersed in an aqueous or hydrophilic solvent. In such a dispersion solution, the concentration of the phosphor nano-particles varies depending upon the strength of the fluorescence, thus it is not particularly limited, but is preferably 0.01 mM to 1000 mM, and is more preferably 0.1 mM to 100 mM. As the dispersion medium, in addition to the above-mentioned alcohols, a hydrophilic organic solvent, such as DMF, DMS, THF, or the like, and water are preferable.

That the surface of the phosphor nano-particle is coated with the surface modifying agent can be identified by using a high-resolution TEM, such as a FE-TEM, or the like, observation of the spacing between particles, and by carrying out chemical analysis.

The phosphor nano-particle coated with the surface modifying agent represented by the formula [I] or [II] can further form a peptide bond by reacting with a molecule in the linker later described through an amidation reaction, or the like, with an amino group, carboxylic group or the like, which is the end group of the surface modifying agent thereof, serving as a reaction group.

The amidation reaction is performed through the condensation between a carboxylic group or a derivative thereof (such as an ester, an acid anhydride, an acid halide, or the like) and the amino group. When an acid anhydride or an acid halide is to be used, it is preferable that a base coexists. When an ester, such as a methylester, an ethylester, or the like, of a carboxylic acid is to be used, it is preferable to carry out heating or evacuation in order to remove the generated alcohol. When the carboxylic group is to be directly amidated, a substance for promoting the amidation reaction, such as an amidation reagent, such as DCC, Morpho-CDI, WSC, or the like; a condensation additive, such as HTB or the like; an active ester agent, such as N-hydroxyphthalimide, p-nitrophenyltrifluoroacetate, 2,4,5-trichlorophenol or the like; or the like may be made to coexist or have been subjected to a prereaction. In addition, it is preferable that for amidation reactions, amino group(s) or carboxylic group(s) of compatible molecules are protected with an adequate protective group in accordance with a conventional method, and deprotected after the reaction.

The phosphor nano-particles which have been bound to the linkers through the amidation reaction are washed and purified by a conventional method, such as gel filtration, or the like, before being dispersed in water or an hydrophilic solvent (preferably, methanol, ethanol, isopropylalcohol, or 2-ethoxyethanol or the like) for use. The concentration of the phosphor nano-particles in this dispersion solution varies depending upon the strength of the fluorescence, thus it is not particularly limited, but it is preferably 1 M to 10-15 M, and is more preferably 0.5 M to 10-10 M.

Magnetic Nano-Particle

The magnetic nano-particles in the present invention are magnetic nano-particles which have an average particle diameter of 2 to 100 nm. Because the average particle diameter is 2 nm or larger, the magnetic nano-particle can be stably manufactured. And the average particle diameter is 100 nm or smaller, thus for example, even when a target substance is inside a cell, the magnetic nano-particle can capture the target substance by entering inside of the cell. The average particle diameter of the magnetic nano-particle is preferably 5 to 100 nm, and is particularly preferably 8 to 80 nm, from the viewpoint of stability and magnetic force.

Such a magnetic nano-particle can be manufactured in accordance with the method as disclosed in, for example, Japanese Patent Publication No. 2002-517085, and the like. For example, an iron oxide or ferrite magnetic nano-particle can be formed by placing an aqueous solution containing an iron (II) compound, or an iron (II) compound and a metal (II) compound in the oxidation condition necessary for formation of a magnetic oxide, and maintaining the pH value of the solution in the range of 7 or over. The magnetic nano-particle of the present invention can also be obtained by mixing an aqueous solution containing a metal (II) compound, and an aqueous solution containing iron (III) in alkaline conditions. Further, the method as disclosed in Biocatalysis, 1991, Vol.5, pp.61 to 69 can also be used.

In the present invention, the preferable magnetic nano-particle is that which is selected from the group consisting of metal oxides, particularly iron oxides and ferrites (Fe,M)₃O₄. Herein, the iron oxides include magnetite, maghemite, hematite or a mixture thereof. The M in the above formula denotes a metallic ion which can form a magnetic metal oxide, being used with the iron ion, and which is exemplarily selected from the transition metals, and is most preferably Zn²⁺, Co²⁺, Mn²⁺, Cu²⁺, Ni²⁺, Mg²⁺, or the like, the molar ratio M/Fe determined in accordance with the stoichiometric composition of the ferrite selected. The metallic salt is supplied in solid or in the form of a solution, however, it is preferably a chloride salt, a bromide salt, or a sulfate.

Among these, iron oxides are preferable from the viewpoint of safety.

For example, in order to form magnetite, it is preferable that iron exist in two different oxidation conditions, i.e., as Fe²⁺ and Fe³⁺ in the solution. The two different oxidation conditions can be provided in the solution by adding a mixture of an iron (II) salt and an iron (H) salt, preferably, the iron (II) salt in a molar quantity slightly greater than that for the iron (II) salt for the composition of the desired magnetic oxide. Or by adding an iron (II) salt or an iron (II) salt for conversion of a part of the Fe²⁺ ions or the Fe³⁺ ions to the other oxidation condition, as required, preferably by oxidation or, depending upon the case, by reduction the different oxidations states can be made present in a solution.

It is preferable that this magnetic metal oxide be aged at a temperature of from 30 deg C. to 100 deg C., preferably at a temperature of from 50 deg C. to 90 deg C.

In order to cause an interaction between metallic ions of various types for formation of a magnetic metal oxide, the pH value of the solution is required to be 7 or higher. The pH value is maintained in the desired range by using an appropriate buffer solution as the aqueous solution in the initial addition of the metallic salt or by adding a base to the solution after the necessary oxidation conditions have been obtained. Once a specific pH value in that range of 7 or over has been selected, it is preferable that the pH value be maintained over the entire process of preparing the magnetic nano-particle, in order to assure that the distribution of sizes of the final product is substantially uniform.

In order to control the particle size of the magnetic nano-particle, a process of giving an added amount of the metallic salt to the solution may be provided. In this case, the following two different modes of operation can be performed. One mode of operation is by stepwise addition, and hereinafter, this mode of operation will be called the stepwise mode of operation, wherein the respective components (the metallic salt, the oxidizing agent, and the base) are successively added to the solution in small doses in a prescribed order, preferably in equal quantities every time, and those processes are repeated as many times as required until a desired size of nano-particle is obtained, the amount of addition at the respective times being that which allows aggregation of the metallic ions within the solution (i.e., at any sites other than the surface of the particle) to be substantially avoided.

The other mode of operation provides a continuous mode of operation, wherein the respective components (the metallic salt, the oxidizing agent, and the base) are continuously added, in a prescribed order, to the solution at a substantially uniform flow rates for each component in order to avoid aggregation of the metallic ions within the solution (i.e., at any sites other than the surface of the particle). By using these stepwise or continuous modes of operation, particles which are provided with a narrow size distribution can be formed.

In addition, by adhering a molecule having a functional group to the magnetic surface of the magnetic nano-particle, it is made possible to bind the later described linker to the magnetic surface of the magnetic nano-particle. Examples of such a molecule include polysaccharides, proteins, peptides, polyamines, polyoxyethylenealkyl carboxylic acid and (ω-silane:Si(OR)₃(CH₂)ₙX (in the formula, R denotes an alkyl substituent having 1 to 6 carbon atoms, n denotes an integer from 1 to 18 (1 and 18 inclusive), and X denotes a functional group selected from a group consisting of NH₂, CN, and SH), and the like.

Fluorescent Coloring Matter

The inorganic phosphor nano-particle in the present invention may be that to the surface of which some other fluorescent coloring matter is further directly bonded. In this case, by exciting the inorganic phosphor nano-particle, energy transfer occurs, which can cause some other fluorescent coloring matter bound to the surface thereof to be excited simultaneously. Herein, the usable fluorescent coloring matter is preferably that which is excited by light in the visible region from the phosphor nano-particle, and can emit fluorescent light in the visible region, and more preferably that which has a maximum of fluorescence spectrum between 400 nm to 800 nm. Examples of such a fluorescent coloring matter include cyanine coloring matters (for example, Cy3, Cy5, and the like in the CyDye™ series), fluorescein coloring matters, rhodamine coloring matters, the Alexa coloring matter series of Invitrogen Corporation, the BODIPY coloring matter series, the TexasRed coloring matter series, azacyanine coloring matters as disclosed in the compound examples I-1 to I-74 in WO No. 01/021624, the disclosure of which is incorporated by reference herein.

When a plurality of fluorescent coloring matters are to be used, it is preferable that the wavelengths of the fluorescences emitted be different from one another, from the viewpoint of that can easy detection of different target substances can be made by the simultaneous measurement of the fluorescences. More preferably, the peaks for the fluorescences emitted are separated from one another by 20 to 250 nm, and particularly preferably, by 40 to 150 nm.

It is preferable that the fluorescent coloring matters used can be identified from one another from the order of energy transfer. As such a combination, Alexa 488, 546, 594, and 647 can be mentioned, for example. The combination is not limited to this, and it is possible to provide identification in the same manner by selecting a second fluorescent coloring matter which has an absorption peak at a wavelength longer by 20 nm or more than the maximum wavelength of the fluorescence spectrum for the shortest wavelength fluorescent coloring matter. When third and fourth coloring matters are to be used, selection of the coloring matters can be carried out in the same manner.

Linker

The inorganic phosphor nano-particle and the magnetic nano-particle in the present invention are linked to each other by a linker. This linker may be any one which can linke between the inorganic phosphor nano-particle and the magnetic nano-particle. It is preferable that, to such a linker, at least one compound be bound selected from the group consisting of compounds (ligands) having an affinity (a bindability) to the target substance and external stimuli responsive compounds. When the target substance is a bio-related molecule, a bio-related molecule may be used for the ligand. For example, when an antigen is adopted as the target compound, an antibody can be used as the ligand.

Bio-Related Molecules

Examples of bio-related molecules include nucleic acids and substances other than nucleic acids, such as antigens and antibodies (monoclonal or polyclonal); peptides, and other proteins (amino acids), and polysaccharides; enzymes and their substrate; further, compounds, such as lipids and the like; and organisms, such as viruses, or bacteria, or parts thereof.

Herein, the term “nucleic acid” can be the narrow sense, covering deoxyribonucleic acids (DNA) and ribonucleic acids (RNA), and also the broad sense, covering PNA (peptide nucleic acids). RNA covers mRNA, tRNA, and rRNA. Further, DNA and RNA cover not only the whole DNA and RNA, but also fragments of DNA and RNA.

Such a linker may exist as a part of the surface of the phosphor nano-particle or magnetic nano-particle or may be that which is directly or indirectly provided on the surface of the phosphor nano-particle and/or magnetic nano-particle. The methods for binding a bio-related molecule as the linker to the phosphor nano-particle are known methods for binding a bio-related molecule to an inorganic material, and a person skilled in the art can easily select an appropriate one from them for implementation. As an example of such a method, a functional group, such as an amino group, a carboxyl group, a hydroxyl group, or the like, in the phosphor nano-particle (a surface modifying agent or the later described external stimuli responsive compound), and a reactive substituent, such as an amino group, a carboxyl group, an active ester group, or the like, in the bio-related molecule, may be caused to be directly bonded to each other with an ionic bond or in a covalent bond, or may be caused to undergo a reaction, after a chemical modification, such as introduction of a linker, or the like, has been carried out. The phosphor nano-particle after the reaction can be refined by a general-purpose separation technique, such as chromatography, electrophoresis, recrystallization, or the like.

When a bio-related molecule is to be adopted as the target substance, respective bio-related molecules providing a pair are previously bound to the magnetic nano-particle and the phosphor nano-particle, respectively, and either one of the nano-particles can also be used as the target substance.

External Stimuli Responsive Compounds

External stimuli responsive compound refers to a compound whose structure is changed in response to an external stimulus, such as heat, pH, electricity (electric charge), light or the like, resulting in being swollen/contracted, or otherwise, dispersed/aggregated in the aqueous solution. As the preferable external stimuli responsive compounds in the present invention, heat responsive macromolecules, with which heat is the stimulating factor, and hydrogen-ion concentration (pH) responsive compounds can be mentioned from the viewpoint of ease of control, and it is preferable that the external stimuli responsive compound be at least one of these. This external stimuli responsive compound can be used as a linker for conjugating the phosphor nano-particle or the magnetic nano-particle. The external stimuli responsive compound can also play the role of conjugating the fluorescent complexes to one another, being contracted in response to an external stimulus. Such a conjugation between fluorescent complexes is called “aggregation” herein. By this aggregation, an aggregate of fluorescent complexes comprising a plurality of magnetic nano-particles is formed, being capable of strongly reacting to an external magnetic field.

When only the external stimuli responsive compound is adopted as the linker, it is preferable to dispose an external stimuli responsive compound on both the inorganic phosphor nano-particle and the magnetic nano-particle, respectively. Thereby, through the contraction under an external stimulus, the inorganic phosphor nano-particle and the magnetic nano-particle can be conjugated with each other with certainty. As a result of this, the fluorescent complex of the present invention is formed, and a plurality of fluorescent complexes are aggregated to form an aggregate. Because this aggregate comprises a plurality of magnetic nano-particles, it can react to an external magnetic force with certainty, as compared to a magnetic nanoparticle which exists alone. In addition, as a special case, by disposing a compound having either an anionic inorganic phosphor nano-particle or magnetic nano-particle, and making the other compound cationic in order to cause an ionic association to occur when both are mixed with each other, a fluorescent complex may be formed.

As heat responsive macromolecules, macromolecules which demonstrate a lower critical solution temperature (LCST) and macromolecules which indicate an upper critical solution temperature (UCST) can be mentioned.

Examples of macromolecules which demonstrate an LCST include derivatives of poly-N-substituted acrylamides, such as a poly-N-isopropylacrylamide, an N,N-diethylacrylamide, or the like, and copolymers thereof; derivatives of poly-N-substituted methacrylamides and copolymers thereof; vinylethers, such as polymethylvinylether, and the like; polypropyleneoxide; polyethyleneoxide; poly(N-vinylalkylamide); copolymerized polymers of these macromolecular monomers which indicate LCST; and a polymerizable biotin derivative monomer (as disclosed in WO 01/09141), and the like.

Examples of macromolecules which demonstrate a UCST include a homopolymer of acryloylglycineamide; copolymers of acryloylglycineamide and polymerizable biotin monomers; copolymers of an acrylamide and N-formylacrylamide; copolymers of an acrylamide and N-acetylacrylamide; a macromolecule mainly consisting of a copolymer of nonionic N-acryloylglycineamide and a biotin derivative (N-methacloyl-N′-biotinylpropylenediamine; MBPDA); a UCST-CV polymer as disclosed in JP-A No. 2002-60436; a macromolecule derivative utilizing the keto-enol tautomerism as disclosed in JP-A No. 11-171928; copolymerized polymers as disclosed in JP-A No. 2000-86729 and WO 01/09141; and the like.

Among these heat responsive macromolecules, macromolecules which demonstrate a UCST are preferable, because they are swollen in the living body, and are contracted at room temperature to exhibit mutual aggregatability, and because they allow measurement when a thermally decomposable substance is used as the target without causing degradation.

In addition, heat responsive macromolecules which are a result of copolymerization of a monomer component having a LCST and a monomer component having a UCST, and thus having both an LCST and UCST (as disclosed in JP-A No. 11-255839), are also preferable.

As pH responsive compounds, the macromolecule derivatives utilizing keto-enol tautomerism as disclosed in JP-A No. 11-171928, and the like can be mentioned.

In addition, compound stimuli responsive macromolecules which can provide LCST and UCST transitions in response to a stimulus, or cause reversible dissolution or precipitation depending upon the pH, as disclosed in JP-A No. 11-255831, and ligand responsive gels as disclosed in JP-A No. 2002-226362 can also be used.

Further, because alkyl carboxylic acids and gelatins have an aqueous solubility which varies depending upon the pH, they can be used as a pH responsive compound.

The external stimuli responsive compound may be disposed on at least a part of the magnetic nano-particle or the inorganic phosphor nano-particle; 10% of the particle surface area or more is preferable; and coating approximately over the entire area is particularly preferable. Coating can be easily performed by polymerizing the above-mentioned respective monomers in the presence of the magnetic nano-particles or the inorganic phosphor nano-particles or by adding the respective nano-particles to a solution in which the external stimuli responsive compound is dispersed.

When a nano-particle is to be coated with an external stimuli responsive compound, it is preferable that the coating thickness be 1 nm to 1 μm, and preferably be 1 nm to 100 nm, from the viewpoint of that, when the nano-particles are dispersed, and are subjected to a magnetic force, the nano-particles will be efficiently collected by that magnetic force with no coalescence or deformation being caused.

The external stimuli responsive compound as the linker can be used concurrently with the above bio-related molecules. In this case, the external stimuli responsive compound can be disposed on at least one of either the magnetic nano-particle and/or the inorganic phosphor nano-particle.

When the external stimuli responsive compound is disposed on the magnetic nano-particle side, the magnetic nano-particles can be directly aggregated with an external stimulus being given. When the external stimuli responsive compound is disposed on the inorganic phosphor nano-particle side, the magnetic nano-particles are indirectly aggregated by the aggregation of the inorganic phosphor nano-particles through the linkers, and thus the inorganic phosphor nano-particles are capable of being subjected to an external magnetic force. The external stimuli responsive compounds disposed on the inorganic phosphor nano-particles and those disposed on the magnetic nano-particle may be the same or different from each other.

Target Substance and Ligand

A ligand for capturing the target substance may be bound, in addition to the linker, to either one of the phosphor nano-particle or the magnetic nano-particle in the present invention,linker. When only an external stimuli responsive compound is adopted as the linker, it is particularly preferable that a ligand be provided.

If the target substance contains a bio-related molecule, then the above-mentioned bio-related molecule are applicable as they are. Ligands which capture these bio-related molecules and form pairs with the target substances can be mentioned.

For example, when a known substance which exists in the cell is used as the target substance, the bio-related substance, such as an antigen, which can capture this target substance is applicable as the ligand. When a DNA-bound protein which exists in a cell is to be detected, a bound DNA sequence or a fragment thereof is applicable as a ligand.

Thereby, using the fluorescent complex of the present invention, a target substance, such as a bio-related molecule or the like, can be efficiently detected.

A plurality of different types of ligand may be provided. Thereby, a plurality of target substances can be detected concurrently.

(B) Fluorescent Particle

The fluorescent particles of the present invention comprise the above-described inorganic phosphor nano-particles and, as a linker, the above-described external stimuli responsive compounds; in other words, the fluorescent particles comprise inorganic phosphor nano-particles with average particle diameters of 1 to 50 nm, and external stimuli responsive compounds disposed on the surfaces of the inorganic phosphor nano-particles.

According to the present invention, the fluorescent particle, which is used for detection of the target substance, has the external stimuli responsive compound disposed on the surface thereof, thus by the external stimuli responsive compound swelling/contracting in response to an external stimulus, the fluorescent particles can be aggregated. Thereby, even if the target substance exists in a minute region of the sample, such as in a cell or the like, the fluorescent particle can enter the minute region, and after it being bound to the target substance, the action of the external stimuli responsive compound can cause the fluorescent particles to be aggregated for easy detection. As a result of this, the target substance in the sample can be efficiently detected with certainty.

It is preferable that, to the fluorescent particle of the present invention, a ligand is bonded for capturing the target substance via the surface modifying agents or external stimuli responsive compounds. The fluorescent particles of the present invention may be bound to magnetic nano-particles through the above-mentioned linkers to form a fluorescent complex.

(C) Fluorescence Detection Method

The detection method of the present invention provides a fluorescence detection method for detecting a target substance existing in a sample using a phosphor, comprising: combining the sample containing the target substance with the above fluorescent complexes that further comprise a ligand to cause binding to the target substance (where furthermore, an external stimuli responsive compound may be disposed on the surface of one of either the magnetic nano-particles or the inorganic phosphor nano-particles) for formation of fluorescent complexes to which the target substance has been bound; applying an external magnetic field to said fluorescent complex in the sample for collecting the fluorescent complexes to which the target substance has been bound; irradiating the collected fluorescent complexes with excitation light for exciting said phosphor nano-particles to cause the phosphor nano-particles to emit fluorescent light; detecting the fluorescence emitted from said fluorescent complexes; and detecting the target substance in the sample on the basis of the fluorescence emission. Further, when the light emission from the inorganic phosphor nano-particle is to be converted to light emission from said fluorescent coloring matter by energy transfer, detecting the fluorescence emitted from that fluorescent coloring matter is also comprised in the detection method.

Another detection method of the present invention comprises: combining the sample containing the target substance with said magnetic nano-particles that further comprise a ligand for causing the target substance to bind thereto (where furthermore, an external stimuli responsive compound may be disposed on the surface thereof) for formation of magnetic nano-particles to which the target substance has been bound; applying an external magnetic field to the above-mentioned magnetic nano-particles in said sample for collecting the magnetic nano-particles to which the target substance has been bound; combining the above-mentioned inorganic phosphor nano-particles, which can mutually bind to the above-mentioned magnetic nano-particles through a linker, with the collected above-mentioned magnetic nano-particles for formation of fluorescent complexes which comprise the magnetic nano-particles, the inorganic phosphor nano-particles, and the linker, and include the target substance; irradiating the above-mentioned fluorescent complexes with excitation light for exciting said phosphor nano-particles to cause the phosphor nano-particle to emit fluorescent light; detecting the fluorescence emitted from said fluorescent complex (also in this case, detecting the fluorescence emission of said fluorescent coloring matter by the energy transfer from the light emission of the inorganic phosphor nano-particle is also included); and detecting the target substance in the sample on the basis of said fluorescence emission. The detection method may comprise in addition, after formation of a fluorescent complex, applying again an external magnetic field to the fluorescent complex for collecting the fluorescent complexes. With the present detection method, the inorganic fluorescent nano-particle may also have an external stimuli responsive compound on the surface thereof.

According to these methods, a target substance in the sample can be captured by the fluorescent complex or the magnetic nano-particle via the ligand, and the fluorescent complexes or the magnetic nano-particles can be easily collected by applying an external magnetic force, thus the fluorescent complex including the target substance can be detected with certainty.

In the formation process, the target substance in the sample is bound to the magnetic nano-particle or the fluorescent complex through the ligand, and in the collection process, an external magnetic field is applied to the magnetic nano-particle or the fluorescent complex.

The external magnetic field used here may be provided by any apparatus, or the like, which can collect magnetic nano-particles by magnetic force, and apparatuses which are generally used for collecting magnetic substances can be used as they are. Generation of such an external magnetic field can be easily implemented by a person skilled in the art.

In the process of fluorescence emission, the phosphor nano-particle is irradiated with excitation light for exciting the inorganic phosphor nano-particle to cause it to emit fluorescent light, and, when fluorescent coloring matter is bound to the inorganic phosphor nano-particle, the fluorescent coloring matter is excited to emit light by the energy transfer from the phosphor nano-particle. Next, in the detection process, this fluorescence emission is detected, and further on the basis of this fluorescence emission, the target substance comprised in the fluorescent complex can be detected. The fluorescence emitted is preferably visible light.

Excitation of the phosphor nano-particle is preferably carried out with ultraviolet light from the viewpoint of the necessity for detecting the signal fluorescence in the visible region (particularly with near-ultraviolet light at 300 to 410 nm from the viewpoint of the necessity for minimizing damage to biosamples).

The fluorescent coloring matter which is excited through the light emission of the phosphor nano-particle is preferably that which emits visible fluorescence, from the viewpoints of separation between the excitation light and the signal fluorescence, utilization of low-cost light sources, and construction of a convenient detection system. The requirements for light emission of the fluorescent coloring matter, and the like are as mentioned above.

For detection of the fluorescence emitted from the fluorescent complex, and detection of the target substance on the basis thereof, the conditions and means which are generally used for this purpose can be applied as they are. Such requirements and means can be easily selected by a person skilled in the art. At this time, as the fluorescence to be used as the object of detection, the fluorescence from the phosphor nano-particle is applicable when only the phosphor nano-particle exists in the fluorescent complex. However, when some other fluorescent coloring matter which is bound to the phosphor nano-particle exists in the fluorescent complex, the fluorescence to be used as the object of detection may also be that from the fluorescent coloring matter.

Herein, when the external stimuli responsive compound is used as the linker, an aggregation process which causes the external stimuli responsive compound to be contracted for aggregating the fluorescent complexes or the magnetic nano-particles is further comprised in the detection method. When an aggregation process is provided, the detection method may be further comprised of a dispersion process which causes the external stimuli responsive compound to be swollen for returning the fluorescent complexes or the magnetic nano-particles to the dispersed condition.

When only an external stimuli responsive compound is adopted as the linker, the aggregation process and the fluorescent complex formation process may be implemented simultaneously. In other words, by contracting the external stimuli responsive compound, the phosphor nano-particle and the magnetic nano-particle can be bound to each other, thereby forming the fluorescent complex.

The respective conditions for carrying out the aggregation process and the dispersion process are determined by the external stimulating factor for the external stimuli responsive compound. In other words, the contraction conditions of the external stimuli responsive compound provide the conditions for aggregation, while the swelling conditions provide the conditions for dispersion. For example, when a UCST type heat responsive macromolecule is used as the external stimuli responsive compound, it is preferable to establish the process conditions such that dispersion occurs at a temperature at or above the UCST temperature (preferably, at least 2 deg C. higher), and the aggregation occurs at a temperature below the UCST temperature (preferably, at least 2 deg C. lower). In addition, when a hydrogen ion concentration responsive compound is used as the external stimuli responsive compound, it is preferable that the aggregation or dispersion be carried out at a pH level at least plus or minus 0.2 away from the critical pH value.

According to these fluorescence detection methods, target substance can be detected with certainty, even if they exists only in a trace amount in the sample.

In addition, the fluorescent particle of the present invention is applicable to the above-mentioned detection method, and also can be used in other fluorescence detection methods for detecting target substances existing in the sample.

Other fluorescence detection methods comprise, for example: combining fluorescent particles of the present invention, that further comprise a ligand for causing the target substance to bind thereto with the sample containing the target substance for formation in the sample of a fluorescent particles to which the target substance has been bound; applying an external stimulus for aggregating the fluorescent particles; irradiating the phosphor nano-particles with excitation light for exciting to cause the phosphor nano-particles to emit fluorescent light; detecting the fluorescence emission from said fluorescent particles; and detecting the target substance in the sample on the basis of the fluorescence emission.

According to this method, the target substance in the sample is captured by the fluorescent complex via the ligand, and by the action of the external stimuli responsive compound, the fluorescent particles are aggregated, thus the fluorescent particle including the target substance can be detected with certainty.

Further, when the light emission from the inorganic phosphor nano-particle is converted to light emission from the above fluorescent coloring matter by energy transfer, a step of detecting the fluorescence emitted from the fluorescent coloring matter is also comprised in the detection method.

The process of forming fluorescent particles including the target substance is implemented by combining the fluorescent particles, to which the ligand has been bound, with the sample. The way in which the fluorescent particles are formed can be selected as appropriate, depending upon the type of the target substance. The ligand is bound to the fluorescent particles, thus when the fluorescent particles approache/contact the target substance, they can easily undergo reaction.

In the aggregation process, an external stimulus, which varies depending upon the type of the external stimuli responsive compound, is applied. Thereby, the external stimuli responsive compound is contracted, resulting in the fluorescent particles being aggregated. After the aggregation process, a dispersion process, which causes the external stimuli responsive compound to be swollen to return the fluorescent particles to the dispersed condition, may be further comprised in the detecting method. As the respective conditions for implementing the aggregation process and the dispersion process, those conditions as given in the description of the fluorescence detection method using the fluorescent complex are directly applicable. Also for the particulars concerning the fluorescence emission process, the particulars as given in the description of the fluorescence detection method using the fluorescent complex are directly applicable .

### EXAMPLES

The present invention will be more specifically described with the following examples, however, the present invention is not limited to these.

### Example 1

(1) Preparation of Magnetic Nano-Particle Dispersion

10.8 g of iron(III) chloride 6-hydrate and 6.4 g of iron(II) chloride 4-hydrate were dissolved respectively in 80 ml of a 1N hydrochloric acid solution, and mixed. While this solution being stirred, 96 ml of aqueous ammonia (28 percent by mass) was added thereinto at a rate of 2 ml per min. Then, after the solution was heated to 80 deg C. for 30 min., 1.8 g of oleic acid was added thereinto, which was followed by further stirring for 20 min. After the solution was cooled to room temperature, the pH value was adjusted to 5.5 with 1N hydrochloric acid. The precipitate obtained by decantation was purified with water. The generation of magnetite (Fe₃O₄) of approximately 12 nm in crystallite size was verified by the X-ray diffraction method. To this precipitate, 100 ml of an aqueous solution in which 2.3 g of polyoxyethylene(4.5)laurylether acetic acid was dissolved was added for dispersion.

(2) Coating of Magnetic Nano-Particles With Heat Responsive Macromolecules

To 4 ml of the magnetite nano-particle dispersion prepared in (1), 2.13 g of N-acryloyl glycine aride, 12.7 mg of N-biotinyl-N′-methacryloyl trimethylene amide, and water, and the solution was stirred at 50 deg C. Further, 0.1 g of potassium persulfate was added, and the solution was stirred at room temperature for 6 hr. A black transparent solution obtained was dialyzed to obtain a colloidal solution of magnetite nano-particles coated with heat responsive macromolecules having an upper critical solution temperature (UCST) of 18 deg C.

(3) Preparation of UCST-Type Macromolecule-Coated Magnetic Nano-Particles to Which Avidin is Bound

Into 0.5 ml of a colloidal solution of the UCST-type macromolecule-coated magnetite nano-particle that was prepared in (2), 5 ml of an avidin solution of 1.0 percent by mass, 1 ml of a 1.0-M sodium phosphate buffer solution (pH 7.0), and 3.5 ml of distilled water were mixed, and then the solution was cooled to 8 deg C. The aggregate was recovered with a magnet, and 2 ml of a 0.1-M sodium phosphate buffer solution (pH 7.0) at 30 deg C. was added to the solution to obtain an avidin-immobilized UCST-type macromolecule-coated magnetite nano-particle dispersion.

(4) Preparation of Phosphor Nano-Particle Dispersion

8.8 of zinc acetate 2-hydrate was dissolved in 400 ml of dehydrated ethanol, and the solution was refluxed at 93 deg C. for 2 hr, during which time 240 ml was lost. 240 ml of dehydrated ethanol was added, and the solution was cooled to room temperature. 18 ml of a methanol solution of tetramethylammonium hydroxide of 25 percent by mass was added, and the solution was stirred for 30 min. 7.2 ml of 3-aminopropyltrimethoxysilane and 2.2 ml of water were added, and the solution was stirred at 60 deg C. for 4 hr. The white precipitate generated was filtered off, and was washed with ethanol before being dried.

XRD and TEM analysis revealed that the precipitate was ZnO nano-particles with an average particle diameter of approximately 4 nm. By element analysis and IR spectral extinction measuring method, it was verified that Si and aminopropyl groups were bound to the surface of the ZnO particles. To the precipitate, water was added to prepare a 2 percent by mass aqueous dispersion. When this dispersion was irradiated with light at 370 nm, it exhibited broad and strong fluorescence with a peak wavelength of 540 nm and a half width of 145 nm.

(5) Preparation of Biotin-Bound Zinc Oxide (ZnO) Nano-Particle

To the aqueous dispersion of ZnO phosphor nano-particles that was prepared in (4), NaHCO₃ was added to give a content of 0.1 percent by mass with a pH level of 7.5. To this, a 1 percent by mass aqueous solution of sulfosuccineimidyl (Trade name:D-biotin; manufactured by DOJINDO LABORATORIES) as the biotin labeling agent was added for carrying out the amidation reaction. By purification by gel filtration, a 10⁻⁴-M aqueous dispersion of ZnO nano-particles to which biotin was bound as a functional molecule was prepared.

(6) Immobilization and Separation of Biotin-Bound ZnO Nano-Particles To/From Avidin-Immobilized UCST-Type Macromolecule-Coated Magnetite Nano-Particles

To a test tube, 1 ml of the avidin-immobilized UCST-type macromolecule-coated magnetite nano-particle dispersion obtained in (3), 0.5 ml of the biotin-bound ZnO phosphor nano-particle obtained in (5), 2 ml of a 0.1-M sodium phosphate buffer solution (pH 7.0), and 9 ml of water were added and well mixed. Thereafter, the solution temperature was lowered to 8 deg C. At this time, an aggregate was produced in the test tube. The generated aggregate was recovered by use of a magnet. Even when the supernatant fluid was irradiated with light at 370 nm, fluorescence with a peak wavelength of 540 nm was not detected, but it was verified that the aggregate, when irradiated with light at 370 nm, emitted fluorescence at 540 nm. It was found that the ZnO nano-particles had been immobilized to the UCST-type macromolecule-coated magnetite nano-particles, through the avidin-biotin bond. The aggregate was redispersed when 2 ml of a 0.1-M sodium phosphate buffer solution (pH 7.0) at 30 deg C. was added thereto.

Thus, the UCST-type macromolecule-coated magnetite nano-particles and the phosphor nano-particles form fluorescent complexex through the avidin-biotin bond, and the fluorescent complexes, after being aggregated by the action of an external responsive macromolecule, can be recovered by means of a magnet. Thus, even if the target substance is only in a trace amount, and exists only in a minute region of a sample, it can be efficiently detected with certainty.

### Example 2

(1) Preparation of Phosphor Nano-Particle Dispersion

21.3 g of sodium bis(2-ethylhexyl)sulfosuccinate (AOT) and 5.2 g of water was added into 150 ml of n-heptane, and mixed, being stirred at 3000 rpm for 10 min with a homogenizer for preparation of a reverse micelle solution I. 133 mg of sodium sulfide 9-hydrate was weighed, added to 20 ml of said reverse micelle solution I, and mixed. This solution is called the solution A.

101 mg of zinc oxide and 13 mg of manganese acetate 4 hydrate were weighed, added to 80 ml of said reverse micelle solution I, and mixed. This solution is called the solution B.

Using a homogenizer, the solution B was stirred at 3000 rpm for 10 min, and into this, the solution A was added, the stirring being continued for an additional 10 min for mixing. A transparent ZnS:Mn colloidal dispersion was formed. Into this, 300 ml of a methanol solution containing 4.5 g of sodium oleate and 5 g of 2-mercaptopropionic acid was added, and weakly stirred before being left to stand. The supernatant fluid was removed by decantation, which was followed by further adding 300 ml of methanol to the solution, gently stirring before leaving it to stand. The supernatant fluid was removed by decantation, and to the precipitate, 17 ml of water was added. An aqueous colloidal dispersion of ZnS:Mn which was surface-modified with sodium oleate and 2-mercaptopropionic acid was obtained.

With an excitation wavelength of 325 nm, the fluorescence spectrum was measured. Orange fluorescent light having a maximum at near 590 nm was observed. The concentration of the phosphor nano-particles in the colloidal solution was 30 mM. The average crystallite size of the generated ZnS:Mn was 4.2 nm on XRD measurement.

(2) Coating of ZnS:Mn Phosphor Nano-Particles With Heat Responsive Macromolecules

To 20 ml of the ZnS:Mn phosphor nano-particle dispersion which was prepared in (1), 0.98 g of N-isopropylacrylamide, 25.4 mg of N-biotinyl-N′-methacryloyl trimethylene amide, and 160 ml of water were added and stirred. Further, 0.2 g of potassium persulfate was added, and the solution was stirred at room temperature for 6 hr. The solution obtained was dialyzed to obtain a colloidal solution of the ZnS:Mn phosphor nano-particles coated with biotin-immobilized heat responsive macromolecules having a lower critical solution temperature (LCST) of 30 deg C.

(3) Preparation of LCST-Type Macromolecule-Coated ZnS:Mn Nano-Particles To Which Avidin is Bound

Into 1 ml of a colloidal solution of the ZnS:Mn nano-particle that was prepared in (2), 0.5 ml of an avidin solution of 1.0 percent by mass, 1 ml of a 1.0-M sodium phosphate buffer solution (pH 7.0), and 4 ml of distilled water were mixed, and then the solution was heated to 38 deg C. At this time, an aggregate was produced in the test tube. The aggregate was recovered with a magnet, and 2 ml of a 0.1-M sodium phosphate buffer solution (pH 7.0) at 20 deg C. was added to the solution to obtain an avidin-immobilized LCST-type macromolecule-coated ZnS:Mn nano-particle dispersion.

(4) Preparation of Magnetic Nano-Particle Dispersion

10.8 g of iron (III) chloride 6-hydrate and 6.4 g of iron(II) chloride 4-hydrate were dissolved respectively in 80 ml of a 1N hydrochloric acid solution, and mixed. While this solution being stirred, 96 ml of aqueous anmonia (28 percent by mass) was added thereinto at a rate of 2 ml per min. Then, after being heated at 80 deg C. for 30 min., the solution was cooled to room temperature. The precipitation obtained by decantation was purified with water. The generation of magnetite (Fe₃O₄) of approximately 12 nm in crystallite size was verified by X-ray diffraction. To this precipitate, 200 ml of an aqueous solution in which 1.5 g of sodium dodecylbenzensulfonate and 5 g of 3-aminopropyltrimethoxysilane were dissolved was added, and dispersed at 60 deg C. for 4 hr. After filtering the solution, 200 ml of water was added to the precipitate for redispersion. The solution was filtered using a 0.2-1 m filter, and the filtrate was used in the following experiment.

(5) Preparation of Magnetite Nano-Particle Dispersion To Which Biotin Is Bound

To the aqueous dispersion of magnetite nano-particles that was prepared in (4), NaHCO₃ was added to give a content of 0.1 percent by mass with a pH level of 7.5. To this, a 1 percent by mass aqueous solution of sulfosuccineimidyl (Trade Name: D-biotin; manufactured by DOJINDO LABORATORIES) as the biotin labeling agent was added for carrying out the amidation reaction. By purification with gel filtration, an aqueous dispersion of the magnetite nano-particles to which biotin was bound as the bio-related molecule was prepared.

(6) Immobilization and Separation of Biotin-Bound Magnetite Nano-Particle To/From Avidin-Immobilized LCST-Type Macromolecule-Coated ZnS:Mn Nano-Particle

To a test tube, 0.5 ml of the avidin-immobilized LCST-type macromolecule-coated ZnS:Mn ultrafine particle dispersion obtained in (3), 0.5 ml of the biotin-bound magnetite ultrafine particle phosphor obtained in (5), 2 ml of a 0.1-M sodium phosphate buffer solution (pH 7.0), and 9 ml of water were added and well mixed. Thereafter, the solution temperature was raised to 38 deg C., and the refined aggregate was recovered by use of a magnet. When the supernatant fluid was irradiated with light at 325 nm, fluorescence with a peak wavelength of 590 nm was not detected, but it was verified that the aggregate, when irradiated with light at 325 nm, emitted fluorescence of 590 nm. It was found that, to the LCST-type macromolecule-coated ZnS:Mn nano-particle, the magnetite nano-particle had been immobilized through the avidin-biotin bond. The aggregate was redispersed when 2 ml of a 0.1-M sodium phosphate buffer solution (pH 7.0) at 20 deg C. was added thereto.

Thus, the LCST-type macromolecule-coated ZnS:Mn phosphor nano-particles and the magnetic nano-particles formed fluorescent complexes through avidin-biotin bonds. The fluorescent complexes, after being aggregated, can be recovered by use of a magnet, thus, if even the target substance is only in a trace amount, and only exists in a minute region, it can be efficiently detected with certainty.

What is claimed is:

1. Fluorescent complexes, each comprising:
a magnetic nano-particle;
an inorganic phosphor nano-particle; and
a linker;
wherein the magnetic nano-particles have an average particle diameter of 2 to 100 nm, the inorganic phosphor nano-particles have an average particle diameter of 1 to 50 nm, and the linker links the magnetic nano-particles with the inorganic phosphor nano-particles.

2. The fluorescent complexes of claim 1, wherein the linker comprises an external stimuli responsive compound.

3. The fluorescent complexes of claim 2, wherein the external stimuli responsive compound is disposed at at least one of the magnetic nano-particles or the inorganic phosphor nano-particles.

4. Fluorescent particles each comprising an inorganic phosphor nano-particle, and an external stimuli responsive compound, wherein the inorganic phosphor nano-particles have an average particle diameter of 1 to 50 nm and the external stimuli responsive compound is disposed on the surface of the inorganic phosphor nano-particles.

5. The fluorescent particles of claim 4, further comprising a linker disposed on the surface of the inorganic phosphor nano-particles.

6. The fluorescent particles of claim 5, wherein the inorganic phosphor nano-particles are bound to magnetic nano-particles through the linker.

7. The fluorescent complexes of claim 1 wherein the half width for light emission of the inorganic phosphor nano-particles is 50 to 200 nm.

8. The fluorescent particles of claim 4, wherein the half width for light emission of the inorganic phosphor nano-particle is 50 to 200 nm.

9. The fluorescent complexes of claim 1, further comprises a ligand for causing a target substance to bind thereto.

10. The fluorescent particles of claim 4, further comprises a ligand for causing a target substance to bind thereto.

11. The fluorescent complexes of claim 1, wherein the inorganic phosphor nano-particle is a metal oxide or a metal sulfide.

12. The fluorescent particles of claim 4, wherein the inorganic phosphor nano-particle is a metal oxide or a metal sulfide.

13. The fluorescent complexes of claim 1, wherein the magnetic nano-particle is an iron oxide or a ferrite.

14. The fluorescent complexes of claim 1, wherein the inorganic phosphor nano-particle is surface-modified by a compound represented by the following formula [II] or a surface modifier which is a decomposition product thereof:
M-(R)₄ [I]
wherein, M denotes a Si or Ti atom, and R an organic group, Rs may be respectively the same or different but at least one of the Rs denotes a group having reactivity to the linker or the ligand.

15. The fluorescent particles of claim 4, wherein the inorganic phosphor nano-particle is surface-modified by a compound represented by the following formula [I] or a surface modifier which is a decomposition product thereof:
M-(R)₄ [I]
wherein, M denotes a Si or Ti atom, and R an organic group, Rs may be respectively the same or different but at least one of the Rs denotes a group having reactivity to the linker or the ligand.

16. The fluorescent complexes of claim 1, wherein the inorganic phosphor nano-particle is surface-modified by a compound represented by the following formula [II]:
HS-L-W formula [II]
wherein, L denotes a bivalent conjugating group, and W denotes COOZ or NH₂, and Z denotes a hydrogen atom, an alkaline metal atom, or NX₄, where X denotes a hydrogen atom or an alkyl group.

17. The fluorescent particles of claim 4, wherein the inorganic phosphor nano-particle is surface-modified by a compound represented by the following formula [II]:
HS-L-W formula [II]
wherein, L denotes a bivalent conjugating group, and W denotes COOZ or NH₂, and Z denotes a hydrogen atom, an alkaline metal atom, or NX₄, where X denotes a hydrogen atom or an alkyl group.

18. The fluorescent complexes of claim 2, wherein the external stimuli responsive compound is at least one of a heat stimuli responsive macromolecule or a pH stimuli responsive compound.

19. The fluorescent particles of claim 4, wherein the external stimuli responsive compound is at least one of a heat stimuli responsive macromolecule or a pH stimuli responsive compound.

20. A fluorescence detection method for detecting a target substance in a sample using a phosphor, comprising:
combining the fluorescent complexes of claim 1, that further comprise a ligand for causing the target substance to bind thereto, with the sample for formation in the sample of fluorescent complexes to which the target substance has been bound;
applying an external magnetic field to the fluorescent complexes in the sample for collecting the fluorescent complexes;
irradiating the collected fluorescent complexes with excitation light for exciting the phosphor nano-particles to cause the phosphor nano-particles to emit fluorescent light;
detecting the fluorescence emitted from the fluorescent complex; and
detecting the target substance in the sample on the basis of the fluorescence emission.

21. The fluorescence detection method of claim 20, wherein at least one of the magnetic nano-particles or the inorganic phosphor nano-particles which constitute the fluorescent complexes have an external stimuli responsive compound on the surface of the particle.

22. A fluorescence detection method for detecting a target substance in a sample using a phosphor, comprising:
combining magnetic nano-particles, comprising a ligand for causing the target substance to bind thereto and having an average particle diameter of 2 to 100 nm, with the sample for formation in the sample of magnetic nano-particles to which the target substance has been bound;
applying an external magnetic field to the magnetic nano-particles in the sample for collecting the magnetic nano-particles to which the target substance has been bound;
combining inorganic phosphor nano-particles, which can be mutually bound to the magnetic nano-particles through a linker and which have an average particle diameter of 1 to 50 nm, with the collected magnetic nano-particles for formation of fluorescent complexes which are the fluorescent complexes of claim 1, and to which the target substance has been bound;
irradiating the fluorescent complexes with excitation light for exciting the phosphor nano-particles to cause the phosphor nano-particles to emit fluorescent light;
detecting the fluorescence emitted from the fluorescent complexes; and
detecting the target substance in the sample on the basis of the fluorescence emission.

23. The fluorescence detection method of claim 22, wherein at least one of the inorganic phosphor nano-particles or magnetic nano-particles has an external stimuli responsive compound on the surface of the particles.

24. A fluorescence detection method for detecting a target substance in a sample using a phosphor, comprising:
combining the fluorescent particles of claim 4, that further comprise a ligand for causing the target substance to bind thereto with the sample for formation of fluorescent particles to which the target substance has been bound;
applying an external stimulus for aggregating the fluorescent particles;
irradiating the phosphor nano-particles with excitation light for exciting it to cause the phosphor nano-particles to emit fluorescent light;
detecting the fluorescence emission from the fluorescent particles; and
detecting the target substance in the sample on the basis of the fluorescence emission.
